(19) United States
(12) Patent Application Publication
(10) Pub. No.: US 2013/0061351 A1
(43) Pub. Date: Mar. 7, 2013

(54) METHOD FOR PRODUCING RECOMBINANT PROTEINS FROM PLANT HAIRY ROOTS

(71) Applicants: Michele Boitel-Conti, Amiens (FR); Yoann Huet, Amiens (FR); Francois Guerineau, Salouel (FR); Jean-Pierre Ele Ekouna, Longueau (FR)

(72) Inventors: Michele Boitel-Conti, Amiens (FR); Yoann Huet, Amiens (FR); Francois Guerineau, Salouel (FR); Jean-Pierre Ele Ekouna, Longueau (FR)

(73) Assignee: Universite De Picardie Jules Verne

(21) Appl. No.: 13/695,690

(22) PCT Filed: Apr. 28, 2011
(86) PCT No.: PCT/EP11/56786

§ 371 Date: Nov. 1, 2012

(30) Foreign Application Priority Data
May 3, 2010 (EP) 10305473.0

(51) Int. Cl. C12N 15/82; A01H 5/00; C07K 14/00; C12N 9/00

(52) U.S. Cl. 800/294; 435/183; 800/298; 530/350

(57) ABSTRACT

The invention relates to a method for producing recombinant proteins from transgenic hairy roots, in particular transgenic hairy roots obtained by transforming plants belonging to the Brassicaceae family with Agrobacterium rhizogenes and/or with Agrobacterium tumefaciens.

### FIELD OF THE INVENTION

The invention relates to a method for producing recombinant proteins from transgenic hairy roots, in particular transgenic hairy roots obtained from plants belonging to the Brassicaceae family.

### BACKGROUND OF THE INVENTION

Proteins are amino-acid biopolymers synthesized by all living organisms. There are involved in virtually all aspects of the cell life. Enzymes drive and regulate the metabolism, structural proteins shape the cell, receptors and signaling proteins integrate environmental changes. Nowadays, proteins are widely used not only in the industrial field (enzymes in washing powder, food additive, paper bleaching agents . . . ) but also for medical applications (vaccines and allergens, hormones, antibodies . . . ). Prior to the development of molecular biology and recombinant DNA technology tools, the sole source of proteins of interest remained the producing organism itself. Indeed, insulin was formerly purified from swine while human growth hormone was extracted from human corpse tissues. Major drawbacks of these approaches were the limited availability of the starting material and the rather low content of the protein of interest. Moreover, the risk of viral contamination concerning proteins with medical applications remained high, especially when they were extracted from human tissues. In the 80′, the recombinant DNA technology provided alternatives to such problems by allowing the overproduction of foreign proteins (recombinant proteins) in a given host organism. Indeed, animal insulin was the first recombinant protein with medical application to be produced in the bacterium E. coli. At the present time, cultures of animal cells and E. coli are the two references for the bioproduction of recombinant proteins.

However, bacteria are unable to produce complex (glycosylated) proteins and animal cell culture is a rather expensive process which cannot exclude the risk of animal virus contamination. Alternative bioproduction systems thus emerged during the last two decades, including plants which are considered to be safe (no viral risk), able to produce complex proteins and cheap to grow. Containment of plant-derived bioproduction systems (in greenhouses for whole plant, or bioreactors for plant cell and organ cultures) is preferred to plants grown in field. Hairy roots are an example of such confined bioproduction system as they can easily be cultured in bioreactors and transgenic clones can be obtained for any gene of interest. This particular root system is generated following infection of the plant cell by Agrobacterium rhizogenes which naturally transfer several bacterial genes (rol genes) into the plant genome. These rol genes force the infected plant cell (from stem, leaf . . . ) to follow a new development program leading to the formation at the infection site of a new root system: the hairy roots. A. rhizogenes can be genetically modified in order to perform the transfer of a gene of interest (encoding a biopharmaceutical) for the production of transgenic hairy roots.

Tobacco (Nicotiana tabacum) is by far the most largely used plant species for the production of recombinant protein but several others were shown to be suitable (Daniell et al., Trends in Plant Science, 2009). It is indeed a well characterized species which is largely used in academic and applied research. Nicotiana tabacum hairy roots were shown to be able to produce and secrete not only the model recombinant protein GFP but also several relevant proteins for therapeutic applications in human health.

For example, previous work by Medina-Bolivar (Medina-Bolivar et al., 2004) showed the accumulation of eGFP in the medium of Nicotiana tabacum root culture at 0.8 mg/L after 21 days.

More recently, Woods et al. (Woods et al. 2008, BMC Biotechnology, 8:95) have produced human acetylcholinesterase from hairy-root organ cultures from Nicotinia benthamiana.

### SUMMARY OF THE INVENTION

The present invention relates to a method for obtaining a recombinant protein from hairy roots comprising the steps of:

a) transforming a plant with a strain of Agrobacterium rhizogenes and/or with a strain of Agrobacterium tumefaciens comprising the rol genes;

and

b) transforming said plant with a vector containing an expression cassette comprising a gene encoding said recombinant protein;

wherein said plant belongs to the Brassicaceae family.

The invention also relates to hairy root cultures obtainable by the transformation steps described above.

The invention also relates to a culture medium containing a recombinant protein obtainable by the method described above.

The invention further relates to a recombinant protein obtainable by the method described above.

Indeed, the inventors have discovered that hairy roots obtained by transforming a plant belonging to the Brassicaceae family with Agrobacterium rhizogenes are capable of secreting large amounts of recombinant protein into the extracellular medium. As an alternative, Agrobacterium rhizogenes can be replaced by Agrobacterium tumefaciens, in the presence of the rol genes, known to induce the formation of hairy roots.

One advantage of the present invention is to simplify the recovery and downstream processing of the recombinant protein. Another advantage is that the root biomass is not destroyed for protein recovery and a given culture can be used for several cycles of production of said recombinant protein. In contrast to plants grown in fields where environmental factors (temperature changes, drought, pest attacks, pesticide/herbicide use . . . ) may considerably affect the production level of the recombinant protein, transgenic root culture conditions in bioreactors are controlled and standardized leading to a homogenous production among different batches. Moreover, roots do not produce pollen and can not survive outside the bioreactor, which eliminates the risk of transgene dissemination in the environment.

Yet another advantage of the method of the invention is the possibility to produce orally-delivered biopharmaceuticals by using plant species which provide edible roots generally found in human/animal diet. There is in this case no need to purify the secreted recombinant protein.

The use of edible plant species for several hundred years in human/animal nutrition is a good indication of their harmless nature. In contrast to tobacco, which has been used extensively in the past for this type of application, and which belongs to the Solanaceae family well known for the ability of several of its species member to produce potentially toxic compounds (alkaloids), including nicotine.

The use of an edible plant root system for the production of therapeutic recombinant proteins thus lowers safety concerns relative to the starting raw material prior to protein purification (no animal virus).

Thus, advantageously, the method of the invention enables the obtaining of high levels of recombinant proteins, with reduced purification/downstream processing costs, enables a new formulation for orally-delivered biopharmaceuticals and lowers safety concerns for human health and the environment

### DETAILED DESCRIPTION OF THE INVENTION

In one aspect, the invention relates to a method for obtaining a recombinant protein from hairy roots comprising the steps of:

a) transforming a plant with a strain of Agrobacterium rhizogenes and/or with a strain of Agrobacterium tumefaciens comprising the rol genes;

and

b) transforming said plant with a vector containing an expression cassette comprising a gene encoding said recombinant protein;

wherein said plant belongs to the Brassicaceae family.

Typically, the recombinant protein according to the invention is secreted into the extracellular medium, due to the presence of a signal peptide in the expression cassette.

In a preferred embodiment, a strain of Agrobacterium rhizogenes is used.

In another embodiment, a strain of Agrobacterium tumefaciens is used. In this embodiment, the strain of Agrobacterium tumefaciens is capable of inducing the formation of hairy roots if it harbors a pRi plasmid comprising the rol genes.

Typically, steps a) and b) are performed simultaneously.

In one embodiment, the strain of Agrobacterium itself contains an expression cassette comprising a gene encoding the recombinant protein.

In another embodiment, step b) is performed prior to step a)

In this embodiment, hairy roots can be obtained by transforming a transgenic plant which expressed the gene of interest with a strain of Agrobacterium under conditions which induce the formation of hairy roots.

Thus, in one embodiment the invention relates to a method as described above, wherein step a) and step b) are performed simultaneously by transforming said plant belonging to the Brassicaceae family with a strain of Agrobacterium rhizogenes, wherein said strain of Agrobacterium rhizogenes contains an expression cassette comprising a gene encoding said recombinant protein.

In other words, the invention relates to a method for obtaining a recombinant protein from hairy roots comprising the step of transforming a plant with a strain of Agrobacterium rhizogenes, wherein said plant belongs to the Brassicaceae family and wherein said strain of Agrobacterium rhizogenes contains an expression cassette comprising a gene encoding said recombinant protein.

As used herein, the expression “plant belonging to the Brassicaceae family” has its general meaning in the art. It encompasses any plant of the Brassicaceae family, also known as the crucifers, the mustard family or cabbage family.

It contains over 330 genera and about 3,700 species, according to the Royal Botanic Gardens, Kew.

The largest genera are Draba (365 species), Cardamine (200 species, but its definition is controversial), Erysimum (225 species), Lepidium (230 species) and Alyssum (195 species.)

Well-known species: Brassica oleracea (cabbage, cauliflower, etc.), Brassica rapa (turnip, Chinese cabbage, etc.), Brassica napus (rapeseed, etc.), Raphanus sativus (common radish), Armoracia rusticana (horseradish), Matthiola (stock), Arabidopsis thaliana (model organism) and many others. Among these species, several produce edible roots (turnip, radish . . . )

In a preferred embodiment, said plant belonging to the Brassicaceae family is selected from the group consisting of Raphanus sativus, Raphanus sativus var. niger, Brassica Oleracea L. Convar, Brassica rapa and Arabidopsis thaliana.

Even more preferably, said plant belonging to the Brassicaceae family is Brassica rapa

Transformation by Agrobacterium rhizogenes and/or Agrobacterium tumefaciens is a known technique in the art. The skilled person is familiar with the different techniques commonly employed for carrying out said transformation step. According to the species to be transformed, different parts of the plant can be used for the infection (hypocotyls, leaves etc.).

Typically, infection by Agrobacterium rhizogenes and/or Agrobacterium tumefaciens is carried out by applying an Agrobacterium rhizogenes and/or Agrobacterium tumefaciens inoculum to plant tissues which has been previously wounded.

Several strains of Agrobacterium rhizogenes can be used for carrying out the invention. Suitable strains include, but are not limited to, the Agrobacterium rhizogenes strain TR7, also known as ATCC 25818 and the strains LBA 9402, A4T, A4, LBA1334, ATCC 11325, ATCC 15834 and LMG 155.

In a preferred embodiment, said strain of Agrobacterium rhizogenes is the strain ATCC 25818.

Several strains of Agrobacterium tumefaciens can be used for carrying out the invention. Suitable strains include, but are not limited to, A. tumefaciens C58, C58C1, LBA4404, GV2260, GV3100, A136, GV3101, GV3850, EHA101, EHA105, AGL-1.

The expression “rol genes” as used herein has its general meaning in the art. It refers to the group of bacterial genes which are capable of inducing the formation of hairy roots (Schmülling et al., 1988; Bulgakov et al., 2008 and references therein). Typically, the rol genes are harbored by a plasmid such as a pRi plasmid.

As used herein, the term “expression cassette” has its general meaning in the art. It refers to a nucleic acid construct, which, when present in a given cell under suitable condition, enables the expression of a gene of interest. According to the present invention, said gene of interest is gene encoding a recombinant protein which one wishes to produce and collect.

The expression cassette can be contained in any suitable expression vector. Typically, the expression may be a binary vector, such as the pRD400 binary vector, which has been modified to include the gene encoding the recombinant protein of interest.

In one embodiment, said expression cassette comprises a promoter, a signal peptide, a gene encoding said recombinant protein and a polyadenylation sequence.

In one embodiment, the promoter is a promoter derived from a Brassicaceae plant-infecting virus (Cauliflower Mosaic Virus).

In a preferred embodiment, said promoter is the CaMV35S promoter.

In one embodiment, said promoter is an heat or nutrient inducible promoter, such as Arabidopsis heat shock protein (Hsp) and transcription factor (Hsf) promoters (William R Swindell et al., 2007) or the promoter of the At2g33830 Arabidopsis gene.

According to one embodiment of the invention, the expression cassette comprises a signal peptide. In one embodiment, said signal peptide is the native signal peptide comprised in the gene encoding the recombinant protein which is to be expressed and secreted. In an alternative embodiment, said signal peptide is derived from a Brassicaceae plant. In a preferred embodiment, said signal peptide is a signal peptide from the Arabidopsis pectin methylesterase AT1G69940 or a variant thereof which differs by the replacement, deletion or addition of one or several amino acids, typically by the addition of 1, 2, 3 or 4 amino acids.

In a preferred embodiment, said signal peptide is the signal peptide as set forth in SEQ ID NO:1.

The method of the invention is suitable for obtaining any type of recombinant protein. As used, the expressions “recombinant protein” has its general meaning in the art. It refers to a protein encoded by a nucleic acid construct obtained through the recombinant DNA technology and transferred into a recipient cell, tissue or organism for the production of the said recombinant protein. The expressions “recombinant protein”, “heterologous protein” or “protein of interest” are used interchangeably. Typically, said recombinant protein is a protein which is usually not expressed by the plant belonging to the Brassicaceae family, or not expressed at significant levels.

In one embodiment, said recombinant protein is a viral protein, such as a protein from the Hepatitis B virus. In a preferred embodiment said recombinant protein is the protein referenced under SwissProt accession number P03141.3 (large envelope protein from Hepatitis B virus genotype A2 subtype adw2).

In another embodiment, said recombinant protein is a protein from an animal species, preferably a mammalian species such as a rodent, a feline, a canine or a primate. Even more preferably, said recombinant protein is a human protein.

Suitable recombinant proteins according to the invention include, but are not limited to, allergens, vaccines, enzymes, enzyme inhibitors, antibodies, antibody fragments, antigens, toxins, anti-microbial peptides, hormones, growth factors, blood proteins (such as albumin, coagulation factors, transferrin), receptors and signaling proteins, protein component of biomedical standards, protein component of cell culture media, fusion or tagged proteins, cystein (disulfide bridges)-rich peptides and proteins, and glycosylated plant proteins (such as lectins, papain . . . ).

In one embodiment, the recombinant protein is not the peroxidase which is usually expressed in the plant belonging to the Brassicaceae family.

In a preferred embodiment, said recombinant protein is selected from the group consisting of monoclonal antibodies and antibody fragments (single chain FV . . . ), antigens especially from bacteria, virus or fungi sources, allergens, immunoregulators, digestive enzymes and orally-delivered proteins with therapeutic use (lipase, pepsine . . . ).

The invention also relates to a hairy root culture obtainable by:

a) transforming a plant with a strain of Agrobacterium rhizogenes and/or with a strain of Agrobacterium tumefaciens comprising the rol genes;

and

b) transforming said plant with a vector containing an expression cassette comprising a gene encoding said recombinant protein;

wherein said plant belongs to the Brassicaceae family.

In one embodiment, the invention relates to a hairy root culture obtainable by transforming a plant with a strain of Agrobacterium rhizogenes, wherein said plant belongs to the Brassicaceae family and wherein said strain of Agrobacterium rhizogenes contains an expression cassette comprising a gene encoding said recombinant protein, wherein said transformation is as defined above.

In one embodiment, the culture medium containing said recombinant is collected and is directly used for future applications.

Thus, the invention relates to a hairy root culture medium which contains a recombinant protein, obtainable by the method described above.

Advantageously, the culture medium containing the recombinant protein according to this embodiment is suitable for oral use in humans and/or animals without any purification step.

In an alternative embodiment, the recombinant protein is obtained after one or several purification steps. Typically, the culture medium can first be clarified by standard filtration or low speed centrifugation in order to remove cell fragments. The protein of interest is then either precipitated by high salt concentration and dialyzed or directly fixed on an affinity chromatography column. The concentrated recombinant protein can then either be lyophilized or stored in an appropriate storage buffer at low temperature.

Suitable protocols adapted to each recombinant protein obtainable according to the invention and to each type of application envisaged are standard techniques in the art, and the skilled person will readily select the appropriate purification step(s), if any, for the desired application.

The invention also relates to a recombinant protein obtainable by the method as defined above. Advantageously, said recombinant protein has a purity of at least 20%, preferably at least 25%, even more preferably 30%, 40%, 50%, 60%, 70%, 80%, 90%, 95%. In particular, it is devoid of animal proteins and animal pathogens (virus, prions . . . ).

The invention will be further illustrated by the following examples. However, these examples should not be interpreted in any way as limiting the scope of the present invention.

### FIGURE LEGENDS

FIG. 1: schematic representation of the 6× His-eGFP encoding pRD400 plasmid (pRD400-gfp).

FIG. 2: 6× His-eGFP quantification in transgenic hairy root tissue of Brassica rapa (VER) and Nicotiana tabacum (Nt) species.

FIG. 3: intracellular eGFP content in the different parts of the cultured root biomass for the VER and the Nt species. The central part (zone 1) contains the oldest tissues while the peripheral part (zone 3) is composed by the young growing root tissues. The zone 2 is an intermediate part. Analysis was performed for root biomass cultured for 20 or 40 days.

FIG. 4: Secretion of 6× His-eGFP over time in VER and Nt root cultures.
(A) 26 μl of Ver or Nt root culture medium were sampled at the indicated times during the course of the culture and analysed 12.5% acrylamide gel. Total secreted proteins were revealed by a coomassie stain. 500 ng of bacterially expressed 6× His-eGFP were loaded on the same gel as a quantitative and qualitative control. M: molecular marker.
(B) Concentration of the secreted 6× His-eGFP of the same samples as the panel A was determined by fluorescence measurements.

FIG. 5: Western blot analysis of intra- and extra-cellular 6× His-eGFP production for VER and TOU root culture.

Total soluble protein extract (intra- or extra-cellular) of TOU and VER 6× His-eGFP-expressing hairy root clones were analyzed by western blot using a primary anti-GFP antibody. 50 μg of soluble proteins were loaded for each total root protein extract. For the detection of secreted 6× His-eGFP, TCA precipitated proteins from 6 ml of the culture medium were loaded. TOU_{WT} is a wild type hairy root clone which was not transformed with the pRD400-gfp construct. TOU₁ and TOU₂ are two independent 6× His-eGFP-expressing clones from the TOU species. TOU₁ is a 21 day-old root culture whereas all other clones were cultivated for 10 days. 100 ng of the commercially available pure 6× His-eGFP were loaded as a quantitative and qualitative control.

FIG. 6: Performance for 6× His-eGFP secretion for hairy root cultures of Nicotiana tabacum (Nt) and the three edible species Brassica rapa (Ver), Spinacia oleracea (MAT) and Daucus carota (TOU), as determined by fluorescence measurements.

FIG. 7: Secreted 6× His-eGFP was quantified by fluorometry in the culture medium of 20 day-old independent clones. The identification number of each independent clone is indicated on the abscise axis. Brassica rapa (VER), Raphanus sativus var. niger (NOI), Raphanus sativus (CER) and Brassica Oleracea L. Convar (QUI) belong to the Brassicaceae family, while Nicotiana tabacum (Nt) belongs to the Solanaceae family. Not represented on this graph are the Daucus carota (TOU) clones from the Apiaceae family which do not produce any detectable level of secreted 6× His-eGFP.

### EXAMPLES

I. Material and Methods

A. Recombinant Binary Plasmid for 6× His-eGFP Expression

The coding sequence of 6× His-eGFP (p6× HIS-EGFP, CLONTECH) was cloned in the Asp718 and BamHI restriction sites of the pRD400 plant expression vector (Datla et al., 1992), in frame with the 6× His-tag and the secretion signal sequence from the Arabidopsis pectin methylesterase AT1G69940. In order to improve signal peptide cleavage, a C-terminal DP dipeptide was added to the naturally occurring signal peptide sequence to lead MGYTNVSILLGLLMVFVTPMVFADP (SEQ ID NO:1). Gene expression is driven by a duplicated CaMV35S promoter for 6× His-eGFP and a Nos promoter for the nptII selectable marker (kanamycin resistance). The resulting plasmid, pRD400-gfp has the sequence set forth in SEQ ID NO:2.

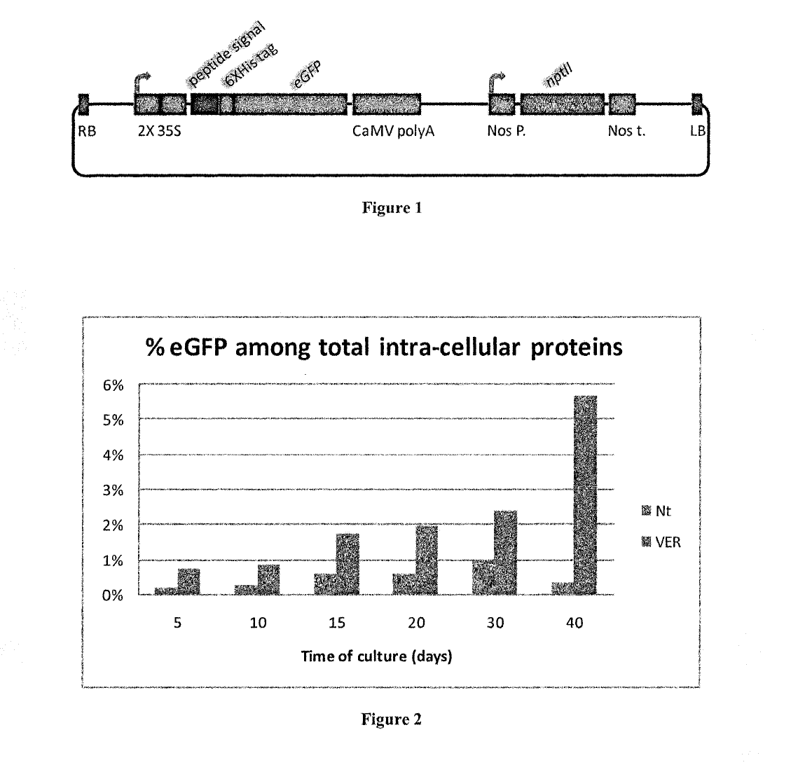

A. rhizogenes strain ATCC 25818 was been transformed by electroporation with the empty or the 6× His-eGFP encoding pRD400 plasmid (pRD400-gfp) (see FIG. 1).

B. Production, Selection and Culture of Transgenic Hairy Roots

1. Plant Species and Culture in Vitro

The following plant families and species (abbreviation in brackets) were used for hairy root production:

| 5 plant families | 17 plant species (species name abbreviation) |
|---|---|
| Apiaceae | Daucus carota (TOU) |
| Asteraceae | Lactuca sativa (BRU) |
| Brassicaceae | Raphanus sativus (CER), Raphanus |
| | sativus var. niger (NOI), Brassica |
| | Oleracea L. Convar (QUI), Brassica rapa (VER) |
| Chenopodiaceae | Spinacia oleracea (MAT) |
| Solanaceae | Nicotiana tabacum (Nt) |

Seeds were purchased from Gondian (www.gondian.com). They were surface sterilized with 70% ethanol for 5 min, 7% bleach for 10 min and washed 5 times with sterile water before to be placed on half strength MS solid medium pH 5.8 supplemented with 1% sucrose. Germination and seedling growth occurred at 22° C. with a 16 h light/8 h dark photoperiod.

2. Plant Infection by Agrobacterium Rhizogenes

We used the Agrobacterium rhizogenes strain TR7 (ATCC 25818) provided by the BCCM™/LMG Bacteria Collection, Laboratorium Voor Microbiologie, Universiteit Gent.

Agrobacterium rhizogenes was grown on MGL plates (2.5 g/l yeast extract, 5 g/l tryptone, 5 g/l mannitol, 5 g/l NaCl, 1.16 g/l Na-glutamate, 0.25 g/l KH2PO4, 0.1 g/l MgSO4, 1.0 mg/l biotin, 8 g/l Agar, pH 7.0) eventually supplemented with 50 mg/l kanamycin for selection of the binary plasmid. Inoculums were prepared from a 20 ml liquid bacterial culture grown overnight at 25° C. in MGL medium. The suspension was centrifuged for 5 min at 15,000 rpm and collected cells were resuspended in fresh MGL medium and diluted to obtain an optical density of 1±0.1 at 600 nm.

Plant infection (except for Nicotiana tabacum and Daucus carota) was performed by pricking with a needle the hypocotyls of 3-10 day old seedlings and by applying the Agrobacterium inoculum with a sterile cotton swab on the injured zone. Depending on the plant species, hairy roots emerged from the wounded site 2 to 5 weeks after infection.

For Nicotiana tabacum, young leaves of 4-week old plant grown aseptically on half strength MS solid medium supplemented with 1% sucrose were cut at the petiole and transferred as a whole piece on half strength MS solid medium pH5.8 supplemented with 3% sucrose. The main central vein was then longitudinally sectioned, starting from the middle of the leaf toward the petiole extremity. Agrobacterium inoculum was then applied on the injured zone as described above. After 4 days, leaves were transferred on solid MS medium supplemented with 3% sucrose and 300 mg/l cefotaxime sodium (MS3cef medium) in order to get rid of the Agrobacteria. Numerous Nicotiana tabacum hairy roots emerged from de middle vein after about 4 weeks.

For Daucus carota, the first 2 leaves of 10-14 day old seedling were injured at the extremity of 3-4 leaflets using a forceps and immediately painted with a sterile cotton swab previously soaked in the Agrobacterium inoculum.

3. Selection and Culture of 6× His-eGFP-Expressing Hairy Root Clones

Infected hypocotyls developing hairy roots were cut out from the seedling and placed on MS3cef medium. After 7-10 days, 15 to 40 independent hairy root tips (except for MAT for which only 5 independent clones were available) were transferred on fresh MS3cef solid medium where they grow for 2-4 weeks. Root tips emerging from Nicotiana tabacum or Daucus carota leaves were treated in a similar manner. For each clone, a root fragment was directly checked for GFP expression by observing fluorescence emission using a Nikon Eclipse 90i microscope. Pictures were taken using a Nikon Digital Sight DS-5Mc camera. For each species, the 10 brightest clones were further cultured in liquid medium.

To initiate cultures in liquid medium, 5 pieces (about 1 cm long) of each hairy root clone were then transferred in a glass tube containing 5 ml of liquid B53cef medium consisting of B5 medium (Duchefa) pH 5.8 supplemented with 3% sucrose and 300 mg/l cefotaxime sodium and cultured for 10 days. Hairy roots were then successively cultured (each time for 3 weeks) in a 100 ml-Erlenmeyer containing 20 ml B53cef and in 250 ml-Erlenmeyer containing 100 ml of B53cef. After those steps aiming to get rid of the Agrobacteria by the use of the antibiotic cefotaxime, the standard culture conditions were the following: 100 ml B5 medium supplemented with 3% sucrose, pH 5.8 at 20-25° C. under dim light, on a Gerhardt RO20 rotator (90 rpm/min). Root clones were sub-cultured every 3 weeks using 1 g of root biomass for 100 ml of culture medium.

Daucus carota hairy roots were cultivated in MW medium (Becard et al., 1988) instead of the B5 medium.

C. 6× His-eGFP Quantification by Fluorometry

For secreted 6× His-eGFP quantification, 50 μl of 1M Tris/HCl pH 8 was added to 500 μl aliquot of the root culture medium. The 6× His-eGFP fluorescence was measured in a BioRad VersaFluo™ fluorometer (excitation filter: 485-495 nm; emission filter: 505-515 nm). Fluorometer calibration was performed using a commercially available recombinant 6× His-eGFP (BioVision) used at a concentration of 1 to 10 mg/L.

For intracellular 6× His-eGFP quantification, root tissue was ground in liquid nitrogen and an aliquot of the obtained tissue powder (equivalent to a spatula tip) was homogenized in 500 μl of ice cold 10 mM Tris/HCl pH8, and centrifuged at 4° C. for 5 min at 14000 g. Total protein quantification of the supernatant was performed using the Bradford method and the sample was diluted with 10 mM Tris/HCl pH8 until to reach a total protein concentration of 50 mg/L. The diluted sample was directly used for fluorescence measurements. The standard curve was performed using a commercially available recombinant 6× His-eGFP (BioVision) diluted in wild-type hairy root extract with a total protein concentration of 50 mg/L.

D. SDS-PAGE and Western Blot

Aliquots of hairy root culture medium were sampled at the indicated times, mixed with ⅓ volume of 3× Laemmli buffer and boiled for 5 min. 40 μl of each sample were loaded on 12.5% acrylamide SDS-PAGE and the gel was stained with coomassie blue. Commercially available 6× His-eGFP expressed in E. coli (Biovision) was used as a quantitative and qualitative control.

Western blot on nitrocellulose membrane (Protean) were performed using the BioRad transfer system according to the manufacturer recommendations.

II. Results

A. Production Level of Intra-Cellular 6× His-eGFP in Nicotiana Tabacum (Nt) and in the Two Edible Brassica Rapa (VER) and Daucus Carota (TOU) Species

1. Microscopic Observation of 6× His-eGFP Fluorescence

Microscopic observation of pRD400-gfp transformed plants, and non-transformed control plants, under white or blue light was carried out. Blue light allowed the observation of the 6× His-eGFP green fluorescence in all three transformed species.

2. Intra-Cellular 6× His-eGFP Quantification by Fluorometry

VER and Nt transgenic hairy root clones showed a similar rate production of intra-cellular 6× His-eGFP during the growth phase (˜the first 30 days). After ˜40 days of culture, Nt hairy roots show a brownish color characteristic of dying tissues and stop to produce the recombinant protein. In contrast, VER roots show a white to slight yellowish color and not only continue to produce 6× His-eGFP but also increase their production rate (FIG. 2).

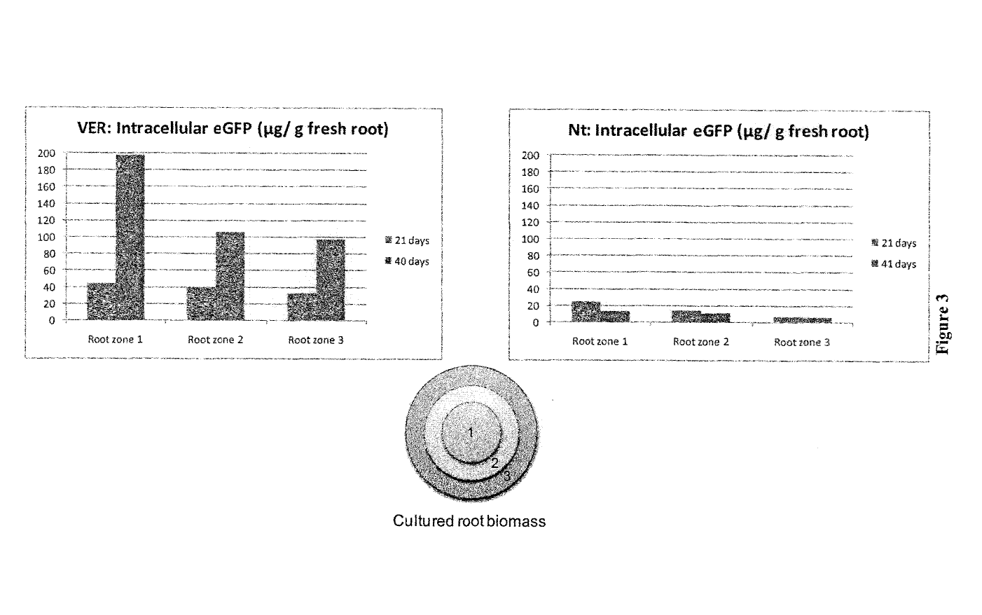

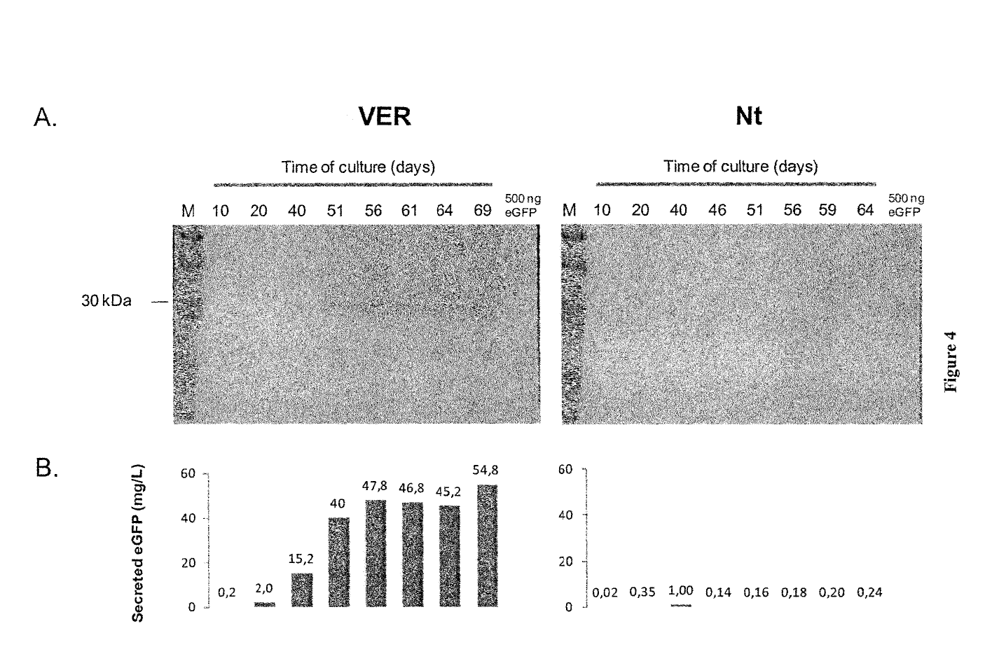

Thus, root aging correlates with the increase in 6× His-eGFP production for the VER species. This was confirmed by the analysis of the different parts of the cultured root biomass (FIG. 3) which is composed by the central zone (zone 1: oldest root tissue), an intermediate one (zone 2), and the peripheral part consisting of very young and proliferating root tissues (zone 3).

B. 6× His-eGFP is Efficiently Secreted by the Brassica Rapa (VER) Species

Accumulation of secreted 6× His-eGFP in the culture medium of Brassica rapa (VER) and Nicotiana tabacum (Nt) hairy root cultures was monitored over time by SDS-PAGE and coomassie staining. The high level of 6× His-eGFP secretion by the VER species allows the direct observation of the recombinant protein on the coomassie-stained acrylamide gel as shown in FIG. 4 (panel A). While secreted 6× His-eGFP is detected in the medium of the VER root culture right from the 10^{th} day, the recombinant protein is undetectable in the Nt root culture medium.

Fluorescence emission of the sampled culture media (FIG. 4, panel B) correlates with the presence of the 26 kDa 6× His-eGFP protein detected on the coomassie-stained gel and confirms its accumulation for the VER root culture over the first 51^{st} days, reaching a concentration of 40 mg/L. Both SDS-PAGE and fluorometric analysis revealed the remarkable stability of 6× His-eGFP in the medium of the VER root culture. In contrast, 6× His-eGFP concentration in the Nt culture medium raised at a maximum of 1 mg/L after 40 days, and decreased dramatically thereafter. This result is in accordance with previous work (Medina-Bolivar et al., 2004) which showed the accumulation of eGFP in the medium of Nt root culture at 0.8 mg/L after 21 days.

C. Brassica Rapa (VER) is Ahead of Several Other Plant Species for its Capacity to Secrete 6× His-eGFP

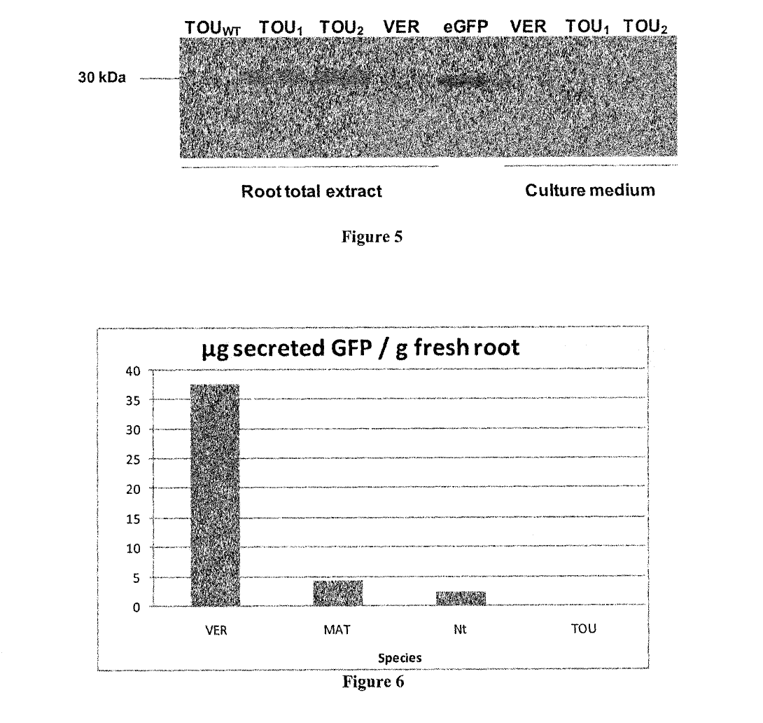

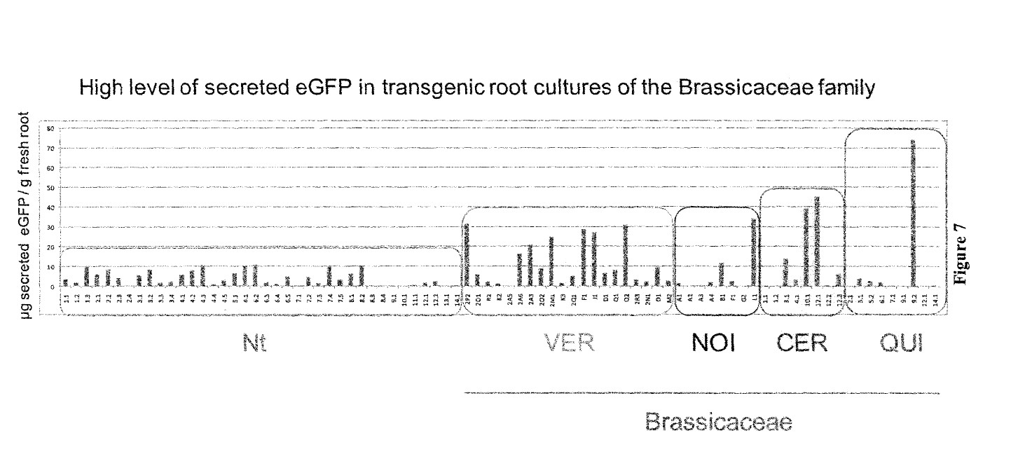

In order to determine if the accumulation at high level of 6× His-eGFP is a specificity of VER, transgenic hairy roots expressing 6× His-eGFP were generated for several other plant species. We first analyzed by western blot the production and secretion of 6× His-eGFP by TOU root cultures in comparison to the previously characterized VER clone (FIG. 5).

The results show that although the TOU species produced similar amounts of intra-tissue 6× His-eGFP, the recombinant protein was undetectable in the culture medium in contrast to the VER root culture. 6× His-eGFP could be detected for both plant species in total intra-cellular soluble protein extract as shown by the revelation of a major band at about 30 kDa, the expected size for 6× His-eGFP. Moreover, the bacterially expressed 6× His-eGFP migrates at the same position. A minor band which likely represents a degradation product was also revealed in these total extracts. The presence of secreted 6× His-eGFP could be clearly detected in the root culture medium of transgenic VER after 10 days of culture but not for the TOU root cultures at either 10 or 21 days of culture, indicating that although efficiently produced in TOU cells, 6× His-eGFP was not secreted by the hairy roots of this plant species.

Fluorescence measurements of the root culture medium of Nicotiana tabacum (Nt) and the three edible species Brassica rapa (Ver), Spinacia oleracea (MAT) and Daucus carota (TOU) also confirm that VER is the most efficient species for the secretion of 6× His-eGFP (FIG. 6).

D. 6× His-eGFP is Efficiently Secreted by the Brassicaceae Family

Other species belonging to the Brassicaceae family were tested for the secretion of 6× His-eGFP.

Secreted 6× His-eGFP was quantified by fluorometry in the culture medium of 20 day-old independent clones. The identification number of each independent clone is indicated on the abscise axis. Brassica rapa (VER), Raphanus sativus var. niger (NOI), Raphanus sativus (CER) and Brassica Oleracea L. Convar (QUI) belong to the Brassicaceae family, while Nicotiana tabacum (Nt) belongs to the Solanaceae family. Not represented on this graph are the Daucus carota (TOU) clones from the Apiaceae family which do not produce any detectable level of secreted 6× His-eGFP.

The most efficient secretion was observed with clones derived from the Brassicaceae family.

Conclusion

In conclusion, these studies demonstrate that is possible to obtain good production and secretion into the extracellular medium of heterologous proteins from hairy roots obtained by transforming plants belonging to the Brassicaceae family.

Similar results were obtained when the protein having the sequence as set forth in SwissProt under accession number P03141.3 was used instead of GFP.

This protein is the large envelope protein from Hepatitis B virus genotype A2 subtype adw2 (strain Rutter 1979) (HBV-A).

### REFERENCES

Becard G, F. J. (1988). Early events of vesicular-arbuscular mycorrhiza formation on Ri T-DNA transformed roots. New Phytol., pp. 211-218.

Bulgakov V P. (2008). Functions of rol genes in plant secondary metabolism. Biotechnology advances, pp. 318-324.

Daniell H, S. N. (2009). Plant-made vaccine antigens and biopharmaceuticals. Trends Plant Sci., pp. 669-679.

Medina-Bolívar F, C. C. (2004). Production of recombinant proteins by hairy roots cultured in plastic sleeve bioreactors. Methods Mol Biol., pp. 351-63.

Scmülling, T., Schell, J., & Spena, A. (1988). Single genes from Agrobacterium rhizogenes influence plant development. EMBO, pp. 2621-2629.

William R Swindell, M. H. (2007). Transcriptional profiling of Arabidopsis heat shock proteins and transcription factors reveals extensive overlap between heat and non-heat stress response pathways. BMC Genomics, p. 125.

Woods R R, G. B. (2008). Hairy-root organ cultures for the production of human acetylcholinesterase. BMC Biotechnol.

1-15. (canceled)

16. A method for obtaining a recombinant protein from hairy roots comprising the steps of:
a) transforming a plant with a strain of Agrobacterium rhizogenes and/or with a strain of Agrobacterium tumefaciens comprising the rol genes;
and
b) transforming said plant with a vector containing an expression cassette comprising a gene encoding said recombinant protein;
wherein said plant belongs to the Brassicaceae family.

17. A method according to claim 16, wherein said expression cassette comprises a signal peptide.

18. A method according to claim 16, wherein step a) and step b) are performed simultaneously by transforming said plant belonging to the Brassicaceae family with a strain of Agrobacterium rhizogenes, wherein said strain of Agrobacterium rhizogenes contains an expression cassette comprising a gene encoding said recombinant protein.

19. A method according to claim 16, wherein said plant belonging to the Brassicaceae family is selected from the group consisting of Raphanus sativus, Raphanus sativus var. niger, Brassica oleracea L. convar and Brassica rapa.

20. A method according to claim 16, wherein said plant belonging to the Brassicaceae family is Brassica rapa.

21. A method according to claim 16, wherein said strain of Agrobacterium rhizogenes is the strain ATCC 25818.

22. A method according to claim 16, wherein said expression cassette comprises a promoter, a signal peptide, a gene encoding said recombinant protein and a polyadenylation sequence.

23. A method according to claim 22, wherein said promoter is a promoter derived from a virus infecting Brassicaceac plants.

24. A method according to claim 22, wherein said promoter is the cauliflower mosaic virus 35S (CaMV35S) promoter.

25. A method according to claim 22, wherein said signal peptide is derived from a Brassicaceae plant.

26. A method according to claim 22, wherein said signal peptide is a signal peptide from the Arabidopsis pectin methylesterase AT1G69940 signal peptide or a variant thereof, such as the signal peptide as set forth in SEQ ID NO:1.

27. A method according to claim 16, wherein said recombinant protein is selected from the group consisting of allergens, vaccines, enzymes, enzyme inhibitors, antibodies, antibody fragments, antigens, toxins, anti-microbial peptides, hormones, growth factors, blood proteins, receptors and signaling proteins, protein component of biomedical standards, protein component of cell culture media, fusion or tagged proteins, cystein (disulfide bridges)-rich peptides and proteins, and plant proteins.

28. A method according to claim 27, wherein said blood proteins are albumin, coagulation factors, or transferrin.

29. A method according to claim 27, wherein said plant proteins are lectins or papain.

30. A hairy root culture obtainable by transforming a plant with a strain of Agrobacterium rhizogenes, wherein said plant belongs to the Brassicaceae family and wherein said strain of Agrobacterium rhizogenes contains an expression cassette comprising a gene encoding said recombinant protein.

31. A culture medium containing a recombinant protein obtainable by the method according to claim 16.

32. A recombinant protein obtainable by the method according to claim 16.


SEQUENCE LISTING (2 sequences)

SEQ ID NO: 1 - DNA, 13583 bp, artificial
  pRD400-GFP
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttaaaacaacaatgggatacacaaatgtgtccattttattaggcctgttgatggtctttgttacaccgatggtgttcgcagatcccatgcatcatcatcatcaccaccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 2 - protein, 25 aa, Arabidopsis thaliana
MGYTNVSILLGLLMVFVTPMVFADP